(19) United States
(12) Patent Application Publication
(10) Pub. No.: US 2017/0218298 A1
(43) Pub. Date: Aug. 3, 2017

(54) PRODUCING RECOVERABLE OIL FROM FERMENTATION PROCESSES

(71) Applicants: DIREVO Industrial Biotechnology GmbH, Cologne (DE); BASF Enzymes, LLC, San Diego, CA (US)

(72) Inventors: Marco Kraemer, Cologne (DE); Vitaly Svetlitchnyi, Cologne (DE)

(21) Appl. No.: 15/501,533

(22) PCT Filed: Jun. 23, 2015
(86) PCT No.: PCT/EP2015/064090

§ 371 Date: Feb. 3, 2017

Related U.S. Application Data
(60) Provisional application No. 62/033,349, filed on Aug. 5, 2014.

(30) Foreign Application Priority Data
Aug. 5, 2014 (EP) 14179841.3

(51) Int. Cl. C11B 1/02; C12P 19/02; C12P 7/14; C12P 19/14

(52) U.S. Cl. CPC C11B 1/025; C12P 19/14; C12P 19/02; C12P 7/14

(57) ABSTRACT

The present technology relates to processes of fermenting a starch-containing material into a fermentation product comprising a fermentation step in the presence of a xylanase in combination with a pectinase on oil partitioning during post-fermentation processing. In particular, the enzyme(s) were added during simultaneous saccharification and fermentation. The finished beer was subjected to beer well incubation, distillation, and then decanting to separate thin stillage from the solids.

### FIELD OF THE INVENTION

The present disclosure relates to an improved process of producing recoverable oil from fermentation processes.

### BACKGROUND OF THE INVENTION

Fermentation products, such as ethanol, are produced by first degrading starch-containing material into fermentable sugars by liquefaction and saccharification and then converting the sugars directly or indirectly into the desired fermentation product using a fermenting organism. Liquid fermentation products such as ethanol are recovered from the fermented mash (often referred to as “beer” or “beer mash”), e.g., by distillation, which separate the desired fermentation product from other liquids and/or solids. The remaining faction, referred to as “whole stillage”, is dewatered and separated into a solid and a liquid phase, e.g., by centrifugation. The solid phase is referred to as “wet cake” (or “wet grains” or “WDG”) and the liquid phase (supernatant) is referred to as “thin stillage”. Dewatered wet cake is dried to provide “Distillers Dried Grains” (DDG) used as nutrient in animal feed. Thin stillage is typically evaporated to provide condensate and syrup (or “thick stillage”) or may alternatively be recycled directly to the slurry tank as “backset”. Condensate may either be forwarded to a methanator before being discharged or may be recycled to the slurry tank. The syrup consisting mainly of limit dextrins and non-fermentable sugars may be blended into DDG or added to the wet cake before drying to produce DDGS (Distillers Dried Grain with Solubles).

Ethanol plants have struggled to maintain profitability, which is highly variable depending upon corn price, demand and price of DDGS, tax credits, gasoline consumption, ethanol exports, and changes to the Renewable Fuels Standard (RFS) mandates. New technologies for energy savings, higher yield of ethanol and higher value for co-products as well as various oil separation technologies contribute to the profitability of producing ethanol.

Corn oil recovery has been recognized as one of the keys in keeping many ethanol plants profitable in times of tight margins by improving operating income and diversifying plant revenue streams. During the past two years, the U.S. ethanol industry has widely implemented advanced corn oil extraction technology. It was estimated that at the end of 2013 about 80% of U.S. ethanol plants were recovering corn oil. According to a report from the U.S. Energy Information Administration⁴, although ethanol facilities with corn oil extraction have slightly higher production costs, their profit margins due to corn oil sales have remained positive and higher than for plants without oil recovery. Due to negative margins, many ethanol plants without oil recovery have chosen to shutdown.

Corn oil recovered as a co-product of ethanol production, also referred to as distiller's corn oil, is an economically attractive and renewable feedstock for biodiesel production. Oil removal from DDGS may also benefit handling and transport of DDGS (less caking and improved flow properties) and expand the use of low-fat DDGS in non-ruminant livestock.

Furthermore, the use of the distillers corn oil, which is inedible feedstock for biodiesel production, would not impact the cost and availability of oil for food.

Reviews of current technologies for corn oil recovery from dry-grind ethanol plants were given in previous reports. Physical pretreatment on dry corn, front- and back-end oil recovery in combination with heating, use of demulsifiers and polar solvents have significantly improved corn oil recovery compared to several years ago. Considerable efforts have been made to use enzymes to facilitate oil separation.

U.S. Pat. No. 6,433,146 discloses extracting oil and zein from corn or corn processing by-products using ethanol. U.S. Pat. No. 7,601,858 discloses a method for recovering oil from a concentrated byproduct, such as thin stillage formed during a dry milling process used for producing ethanol. The method includes forming a concentrate from the byproduct, e.g., by evaporating the by-product, and recovering oil from the concentrate. U.S. Pat. No. 7,608,729 discloses a method of freeing the bound oil present in whole stillage and thin stillage by heating the stillage to a temperature sufficient to at least partially separate, or bind, the oil from the stillage. U.S. Application Publication No. 2010/0058649 discloses a method of separating an oil fraction from a fermentation product, adjusting the pH of the oil fraction, and recovering the oil from the oil fraction.

It is an object of the present disclosure to provide improved methods for increasing the amount of recoverable oil from fermentation processes.

### SUMMARY OF THE DISCLOSURE

The present invention relates to processes of fermenting a starch-containing material into a fermentation product comprising a fermentation step in the presence of a xylanase in combination with a pectinase on oil partitioning during post-fermentation processing. In particular, the enzyme(s) were added during simultaneous saccharification and fermentation. The finished beer was subjected to beer well incubation, distillation, and then decanting to separate thin stillage from the solids.

In one aspect, the present disclosure pertains to a process of producing recoverable oil from fermentation processes, wherein liquefied whole grain mash is thinned by treatment with an efficient amount of enzyme activity of a xylanase in combination with a pectinase.

In a further aspect, the present disclosure pertains to a process for producing recoverable oil from fermentation processes, wherein the process sequentially comprises the following steps: a) milling whole grain; b) liquefying the gelatinised milled whole grain, in the presence of an alpha-amylase; c) saccharifying the liquefied material in the presence of a glucoamylase; d) fermentation with a micro-organism; e) distillation of fermented and saccharified material, providing an ethanol fraction, wherein the liquefied mash is thinned by subjection to an effective amount enzyme activity of a xylanase and a pectinase.

### BRIEF DESCRIPTION OF THE DRAWING

FIG. 1 schematically shows an ethanol production process.

### DESCRIPTION OF THE INVENTION

The object of the present invention is to provide improved methods of increasing the amount of oil recovered from a process for producing a fermentation product.

The addition of a xylanase in combination with a pectinase enhances the oil extraction from thin stillage or syrup following fermentation, which can be used in biodiesel or other biorenewable product production.

In an embodiment, the present invention relates to a process for producing recoverable oil from fermentation processes, wherein the process comprises the steps of:

(a) liquefying a starch-containing material in the presence of an alpha-amylase;

(b) saccharifying the liquefied material obtained in step (a); and

(c) fermenting using a fermenting organism in the presence of a xylanase and a pectinase,

(d) distilling the fermentation product to form a whole stillage;

(e) separating the whole stillage into thin stillage and wet cake; and

(f) recovering the oil from the thin stillage.

In a further embodiment, the present invention relates to a process of recovering oil, comprising

(a) converting a starch-containing material into dextrins with an alpha-amylase;

(b) saccharifying the dextrins using a carbohydrate source generating enzyme to form a sugar;

(c) fermenting the sugar in a fermentation medium into a fermentation product using a fermenting organism, wherein the fermentation medium comprises a xylanase and a pectinase;

(d) distilling the fermentation product to form a whole stillage;

(e) separating the whole stillage into thin stillage and wet cake; and

(f) recovering the oil from the thin stillage.

Stillage is the product which remains after the mash has been converted to sugar, fermented and distilled into ethanol. Stillage can be separated into two fractions, such as, by centrifugation or screening: (1) wet cake (solid phase) and (2) the thin stillage (supernatant). The solid fraction or distillers' wet grain (DWG) can be pressed to remove excess moisture and then dried to produce distillers' dried grains (DDG). After ethanol has been removed from the liquid fraction, the remaining liquid can be evaporated to concentrate the soluble material into condensed distillers' solubles (DS) or dried and ground to create distillers' dried solubles (DDS). DDS is often mixed with DDG to form distillers' dried grain with solubles (DDGS). DDG, DDGS, and DWG are collectively referred to as distillers' grain(s).

In one embodiment of the present disclosure enzymes were added during and/or after the fermentation in the production process to the fermented mash and/or the fermentation medium and before the separation step like distillation, where the desired fermentation main product is separated from the rest of the fermented mash. The enzymes according to the present disclosure were capable of degrading components in the fermented mash (beer or beer mash) and/or the fermentation medium.

The phrase “fermentation media” or “fermentation medium” refers to the environment in which fermentation is carried out and comprises the fermentation substrate, that is, the carbohydrate source that is metabolized by the fermenting organism(s).

The fermentation medium may comprise other nutrients and growth stimulator(s) for the fermenting organism(s). Nutrient and growth stimulators are widely used in the art of fermentation and include nitrogen sources, such as ammonia; vitamins and minerals, or combinations thereof. Recovery Subsequent to fermentation, the fermentation product may be separated from the fermentation medium. The fermentation medium may be distilled to extract the desired fermentation product or the desired fermentation product may be extracted from the fermentation medium by micro or membrane filtration techniques. Alternatively, the fermentation product may be recovered by stripping. Methods for recovery are well known in the art.

Surprisingly, the amount of recoverable oil is increased. DDGS following an ethanol production process from corn typically contains about 13% oil, 31% protein and 56% carbohydrates and other components. Removal of some of the oil from the DDGS will improve the quality of the DDGS for the feed market as many feed producers prefer less oil and fat in the DDGS to make high quality feed.

The starch-containing material may be obtained from cereals. Suitable starch-containing material includes corn (maize), wheat, barley, cassava, sorghum, rye, triticale, potato, or any combination thereof.

Corn is the preferred feedstock, especially when the fermentation product is ethanol. The starch-containing material may also consist of or comprise, e.g., a side stream from starch processing, e.g., C6 carbohydrate containing process streams that may not be suited for production of syrups. Beer components include fiber, hull, germ, oil and protein components from the starch-containing feedstock as well as non-fermented starch, yeasts, yeast cell walls and residuals. Production of a fermentation product is typically divided into the following main process stages: a) Reducing the particle size of starch-containing material, e.g., by dry or wet milling; b) Cooking the starch-containing material in aqueous slurry to gelatinize the starch, c) Liquefying the gelatinized starch-containing material in order to break down the starch (by hydrolysis) into maltodextrins (dextrins); d) Saccharifying the maltodextrins (dextrins) to produce low molecular sugars (e.g., DP1-2) that can be metabolized by a fermenting organism; e) Fermenting the saccharified material using a suitable fermenting organism directly or indirectly converting low molecular sugars into the desired fermentation product; f) Recovering the fermentation product, e.g., by distillation in order to separate the fermentation product from the fermentation mash.

As mentioned above beer (or fermented mash) is the fermentation product consisting of ethanol, other liquids and solids of a desired fermentation product. According to the invention the fermentation product may be any fermentation product, including alcohols (e.g., ethanol, methanol, butanol, 1,3-propanediol); organic acids (e.g., citric acid, acetic acid, itaconic acid, lactic acid, gluconic acid, gluconate, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B12, beta-carotene); and hormones. Fermentation is also commonly used in the production of consumable alcohol (e.g., spirits, beer and wine), dairy (e.g., in the production of yogurt and cheese), leather, and tobacco industries. In a preferred embodiment the fermentation product is a liquid, preferably an alcohol, especially ethanol. The beer contemplated according to the invention may be the product resulting from a fermentation product production process including above mentioned steps a) to f). However, the beer may also be the product resulting from other fermentation product production processes based on starch- and/or lignocellulose containing starting material.

The fermenting organism may be a fungal organism, such as yeast, or bacteria. Suitable bacteria may e.g. be Zymomonas species, such as Zymomonas mobilis and E. coli. Examples of filamentous fungi include strains of Penicillium species. Preferred organisms for ethanol production are yeasts, such as e.g. Pichia or Saccharomyces. Preferred yeasts according to the disclosure are Saccharomyces species, in particular Saccharomyces cerevisiae or baker's yeast.

Furthermore, the use of the enzyme compositions according to the present disclosure in the beer mash after the fermentation and before the distillation process can reduce the viscosity of the beer mash through the degradation of fibers and/or the fermentative microorganisms in the beer. The reduction of the fibers in the beer results in a reduction of the fiber content in the by-products. The early degradation of the NSP's has a direct influence on the separation and the drying conditions of the by-products like DDGS in the production process. The lower viscosity results in lower drying temperatures and also in a shorter drying time resulting in an improved quality of the by-products. For example, the temperature sensitive products like proteins and amino acids are not destroyed.

Further, by adding the enzymes according to the present disclosure to the fermented mash before the distillation step is an advantage since the enzymes in the enzyme compositions are inactivated during the distillation.

Processes for producing fermentation products, such as ethanol, from a starch or lignocellulose containing material are well known in the art. The preparation of the starch-containing material such as corn for utilization in such fermentation processes typically begins with grinding the corn in a dry-grind or wet-milling process. Wet-milling processes involve fractionating the corn into different components where only the starch fraction enters into the fermentation process. Dry-grind processes involve grinding the corn kernels into meal and mixing the meal with water and enzymes. Generally two different kinds of dry-grind processes are used. The most commonly used process, often referred to as a “conventional process,” includes grinding the starch-containing material and then liquefying gelatinized starch at a high temperature using typically a bacterial alpha-amylase, followed by simultaneous saccharification and fermentation (SSF) carried out in the presence of a glucoamylase and a fermentation organism. Another well-known process, often referred to as a “raw starch hydrolysis” process (RSH process), includes grinding the starch-containing material and then simultaneously saccharifying and fermenting granular starch below the initial gelatinization temperature typically in the presence of an acid fungal alpha-amylase and a glucoamylase.

In a process for producing ethanol from corn, following SSF or the RSH process the ethanol is distilled from the whole mash after fermentation. The resulting ethanol-free slurry, usually referred to as whole stillage, is separated into solid and liquid fractions (i.e., wet cake and thin stillage containing about 35 and 7% solids, respectively). The thin stillage is often condensed by evaporation into a thick stillage or syrup and recombined with the wet cake and further dried into distillers' dried grains with solubles distillers' dried grain with solubles (DDGS) for use in animal feed.

In an embodiment of the present disclosure the xylanase may preferably be of microbial origin, such as of fungal origin (e.g., Aspergillus, Fusarium, Humicola, Meripilus, Trichoderma) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. Examples of xylanases useful in the methods of the present invention include, but are not limited to, Aspergillus aculeatus xylanase (GeneSeqP:AAR63790; WO 94/21785), Aspergillus fumigatus xylanases (WO 2006/078256), and Thielavia terrestris NRRL 8126 xylanases (WO 2009/079210). The xylanase may preferably be an endo-1 ,4-beta-xylanase, more preferably an endo-1 ,4-beta-xylanase of GH 10 or GH 11. Examples of commercial xylanases include SHEARZYME(™), BIOFEED WHEAT(™), HTec and HTec2 from Novozymes A/S, Denmark.

Examples of beta-xylosidases useful in the methods of the present invention include, but are not limited to, Trichoderma reesei beta-xylosidase (UniProtKB/TrEMBL accession number Q92458), Talaromyces emersonii (SwissProt accession number Q8X212), and Neurospora crassa (SwissProt accession number Q7SOW4).

Examples of suitable bacterial xylanases include xylanases derived from a strain of Bacillus, such as Bacillus subtilis, such as the one disclosed in U.S. Pat. No. 5,306,633 or Contemplated commercially available xylanases include SHEARZYM E (™), BIOFEED WHEAT(™), (from Novozymes AJS), Econase CE™ (from AB Enzymes), Depol 676™ (from Biocatalysts Ltd.) and SPEZYME(™) CP (from Genencor Int.).).

Xylanase may be added in an amount effective in the range from 0.16×10⁶-460×10⁶ Units per ton beer mash or fermentation medium.

Example of determination of Xylanase Activity (FXU):

The endoxylanase activity is determined by an assay, in which the xylanase sample is incubated with a remazol-xylan substrate (4-O-methyl-D-glucurono-D-xylan dyed with Remazol Brilliant Blue R, Fluka), pH 6.0. The incubation is performed at 50° C. for 30 min. The background of non-degraded dyed substrate is precipitated by ethanol. The remaining blue colour in the supernatant is determined spectrophotometrically at 585 nm and is proportional to the endoxylanase activity.

The endoxylanase activity of the sample is determined relatively to an enzyme standard.

The pectinase used in the methods according to the present disclosure may be any pectinase, in particular of microbial origin, in particular of bacterial origin, such as a pectinase derived from a species within the genera Bacillus, Clostridium, Pseudomonas, Xanthomonas and Erwinia, or of fungal origin, such as a pectinase derived from a species within the genera Trichoderma or Aspergillus, in particular from a strain within the species A. niger and A. aculeatus. Contemplated commercially available pectinases include Pectinex Ultra-SPL™ (from Novozymes), Pectinex Ultra Color (from Novozymes), Rohapect Classic (from AB Enzymes), Rohapect 10 L (from AB Enzymes). Pectinase may be added in an amount effective in the range from 1.4×10⁹-23500×10⁹ Units per ton beer mash or fermentation medium.

Example of determination of Pectintranseliminase Unit (PECTU):

The method is based on the enzyme's degradation of a pectin solution by a transeliminase reaction, the double bonds formed result in an increase in the absorption at 238 nm which is followed by a spectrophotometer.

Reaction conditions

Temperature: 30° C.±0.5° C.

pH: 3.50±0.02

Substrate: 0.24% Pectin (Obipektin, Brown Ribbon Pure, Art. no. 1.1B00.A. Lot no. 0304)

Enzyme concentration: 1.9-2.3 PECTU/mL

Reaction time: 6 minutes

Measuring time: 5 minutes

Wavelength: 238 nm

The activity is determined relative to a PECTU standard. The result is given in the same units as for the standard, which is designated: PECTU—Pectintranseliminase Unit.

The term “alpha-amylase” means an alpha-1,4-glucan-4-glucanohydrolase (E.C. 3.2.1.1) that catalyzes the hydrolysis of starch and other linear and branched 1,4-glucosidic oligo- and polysaccharides.

In an embodiment, the xylanase may be added in an amount of 1-30, e.g., 5-30 7-25,10-20, 10-17, or 12-15 micrograms/g dry solids.

In an embodiment, the pectinase may be added in an amount of 0.01-1.0, e.g., 0.015-0.08, 0.015-0.06, 0.015-0.04, or 0.02-0.03 FXU/g dry solids.

The saccharification and fermentation steps may be carried out either sequentially or simultaneously. The xylanase and the pectinase may be added during saccharification and/or after fermentation when the process is carried out as a sequential saccharification and fermentation process and before or during fermentation when steps (b) and (c) are carried out simultaneously (SSF process).

As mentioned above, the fermenting organism is preferably yeast, e.g., a strain of Saccharomyces cerevisiae or Saccharomyces diastaticus. In an avantegeous embodiment a yeast strain of Saccharomyces diastaticus is used (SIHA Amyloferm®, E. Begerow GmbH&Co, Langenlonsheim, Germany) since their exo-amylase activity can split liquid starch and also dextrin, maltose and melibiose.

In the liquefaction step the gelatinized starch (downstream mash) is broken down (hydrolyzed) into maltodextrins (dextrins). To achieve starch hydrolysis a suitable enzyme, preferably an alpha-amylase, is added. Liquefaction may be carried out as a three-step hot slurry process. The slurry is heated to between 60-95° C., preferably 80-85° C., and an alpha-amylase may be added to initiate liquefaction (thinning). Then the slurry may be jet-cooked at a temperature between 95-140° C., preferably 105-125° C., for about 1-15 minutes, preferably for about 3-10 minutes, especially around about 5 minutes. The slurry is cooled to 60-95° C. and more alpha-amylase may be added to complete the hydrolysis (secondary liquefaction). The liquefaction process is usually carried out at a pH of 4.0 to 6.5, in particular at a pH of 4.5 to 6.

The saccharification step and the fermentation step may be performed as separate process steps or as a simultaneous saccharification and fermentation (SSF) step. The saccharification is carried out in the presence of a saccharifying enzyme, e. g. a glucoamylase, a beta-amylase or maltogenic amylase. Optionally a phytase and/or a protease is added.

Saccharification may be carried out using conditions well known in the art with a saccharifying enzyme, e.g., beta-amylase, glucoamylase or maltogenic amylase, and optionally a debranching enzyme, such as an isoamylase or a pullulanase. For instance, a full saccharification process may last up to from about 24 to about 72 hours, however, it is common to do a pre-saccharification for typically 40-90 minutes at a temperature between 30-65° C., typically about 60° C., followed by complete saccharification during fermentation in a simultaneous saccharification and fermentation process (SSF process). Saccharification is typically carried out at a temperature from 20-75° C., preferably from 40-70° C., typically around 60° C., and at a pH between 4 and 5, normally at about pH 4.5.

The most widely used process to produce a fermentation product, especially ethanol, is the simultaneous saccharification and fermentation (SSF) process, in which there is no holding stage for the saccharification, meaning that a fermenting organism, such as a yeast, and enzyme(s), including the hemicellulase(s) and/or specific endoglucanase(s), may be added together. SSF is typically carried out at a temperature from 25° C. to 40° C., such as from 28° C. to 35° C., from 30° C. to 34° C., preferably around about 32° C. In an embodiment, fermentation is ongoing for 6 to 120 hours, in particular 24 to 96 hours.

During and/or after the fermentation, the fermented mash is subjected to an enzyme composition according to the present disclosure. In an embodiment, the enzyme composition comprises a xylanase and a pectinase.

In a particular embodiment, the process of the present disclosure further comprises, prior to liquefying the starch-containing material the steps of:
reducing the particle size of the starch-containing material, preferably by milling; and
forming a slurry comprising the starch-containing material and water.

The aqueous slurry may contain from 10-55 w/w % dry solids (DS), preferably 25-45 w/w % dry solids (DS), more preferably 30-40 w/w % dry solids (DS) of the starch-containing material. The slurry is heated to above the gelatinization temperature and an alpha-amylase, preferably a bacterial and/or acid fungal alpha-amylase, may be added to initiate liquefaction (thinning). The slurry may be jet-cooked to further gelatinize the slurry before being subjected to an alpha-amylase in step (a).

In a preferred embodiment, the starch containing material is milled cereals, preferably barley or corn, and the methods comprise a step of milling the cereals before step (a). In other words, the disclosure also encompasses methods, wherein the starch containing material is obtainable by a process comprising milling of cereals, preferably dry milling, e. g. by hammer or roller mils. Grinding is also understood as milling, as is any process suitable for opening the individual grains and exposing the endosperm for further processing. Two processes of milling are normally used in alcohol production: wet and dry milling. The term “dry milling” denotes milling of the whole grain. In dry milling the whole kernel is milled and used in the remaining part of the process Mash formation. The mash may be provided by forming a slurry comprising the milled starch containing material and brewing water. The brewing water may be heated to a suitable temperature prior to being combined with the milled starch containing material in order to achieve a mash temperature of 45 to 70° C., preferably of 53 to 66° C., more preferably of 55 to 60° C. The mash is typically formed in a tank known as the slurry tank.

Subsequent to fermentation the fermentation product may be separated from the fermentation medium. The slurry may be distilled to extract the desired fermentation product or the desired fermentation product from the fermentation medium by micro or membrane filtration techniques. Alternatively the fermentation product may be recovered by stripping. Methods for recovering fermentation products are well known in the art. Typically, the fermentation product, e.g., ethanol, with a purity of up to, e.g., about 96 vol. % ethanol is obtained.

Following the completion of the fermentation process, the material remaining is considered the whole stillage. As used herein, the term “whole stillage” includes the material that remains at the end of the fermentation process both before and after recovery of the fermentation product, e.g., ethanol. The fermentation product can optionally be recovered by any method known in the art. In one embodiment, the whole stillage is separated or partitioned into a solid and liquid phase by one or more methods for separating the thin stillage from the wet cake. Such methods include, for example, centrifugation and decanting. The fermentation product can be optionally recovered before or after the whole stillage is separated into a solid and liquid phase.

Thus, in one embodiment, the methods of the disclosure further comprise distillation to obtain the fermentation product, e.g., ethanol. The fermentation and the distillation may be carried out simultaneously and/or separately/sequentially; optionally followed by one or more process steps for further refinement of the fermentation product.

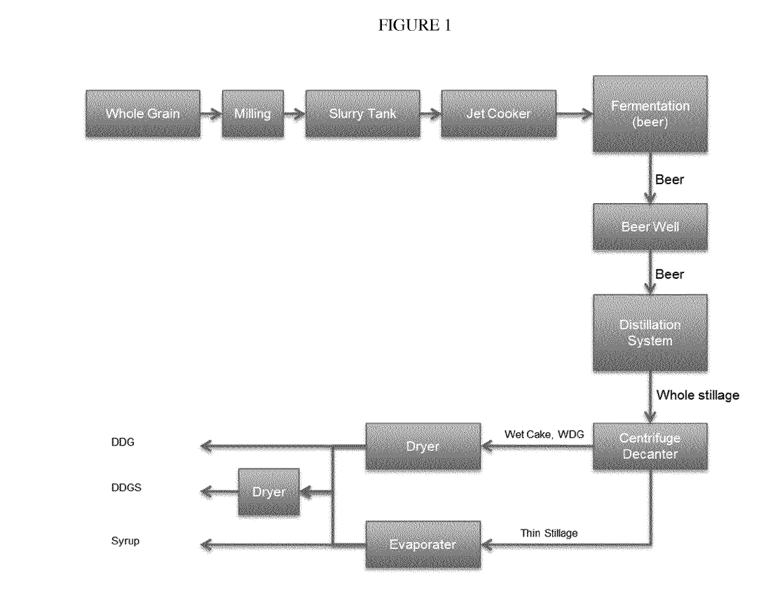

In an embodiment, the aqueous by-product (whole stillage) from the distillation process is separated into two fractions, e.g., by centrifugation: wet grain (solid phase), and thin stillage (supernatant). In another embodiment, the methods of the disclosure further comprise separation of the whole stillage produced by distillation into wet grain and thin stillage; and recycling thin stillage to the starch containing material prior to liquefaction. In one embodiment, the thin stillage is recycled to the milled whole grain slurry. The wet grain fraction may be dried, typically in a drum dryer. The dried product is referred to as distillers dried grains, and can be used as mentioned above as high quality animal feed. The thin stillage fraction may be evaporated providing two fractions (see FIG. 1 and FIG. 2), (i) a condensate fraction of 4-6% DS (mainly of starch, proteins, oil and cell wall components), and (ii) a syrup fraction, mainly consisting of limit dextrins and non-fermentable sugars, which may be introduced into a dryer together with the wet grains (from the whole stillage separation step) to provide a product referred to as distillers dried grain with solubles, which also can be used as animal feed. Thin stillage is the term used for the supernatant of the centrifugation of the whole stillage. Typically, the thin stillage contains 4-6% DS (mainly starch and proteins) and has a temperature of about 60-90° C. In another embodiment, the thin stillage is not recycled, but the condensate stream of evaporated thin stillage is recycled to the slurry containing the milled whole grain to be jet cooked.

Further details on how to carry out liquefaction, saccharification, fermentation, distillation, and recovering of ethanol are well known to the skilled person.

Methods for dewatering stillage and for extracting oil from a fermentation product are known in the art. These methods include decanting or otherwise separating the whole stillage into wet cake and thin stillage. See, e.g., U.S. Pat. Nos. 6,433,146, 7,601,858, and 7,608,729, and U.S. Application Publication No. 2010/0058649. Furthermore, the thin stillage can be evaporated or condensed into syrup or thick stillage from which the oil can be extracted utilizing centrifugation, filtering, heat, high temperature, increased pressure, or a combination of the same. Another way to extract oil is to lower the pH of the thin stillage or syrup. The use of surfactants to break emulsions also enhances oil extraction. Presses can also be used for dewatering. In one embodiment of the disclosure, the presence of pectinase and xylanase in the fermented mash after the fermentation increases the amount of oil in the thin stillage and further the syrup or thick stillage.

The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation as mentioned above. Ethanol with a purity of up to about 96 vol. % can be obtained, which can be used as, for example, fuel ethanol, drinking ethanol, i.e., potable neutral spirits, or industrial ethanol.

The inventions described and claimed herein are not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. Various references are cited herein, the disclosures of which are incorporated by reference in their entireties. The present invention is further described by the following examples which should not be construed as limiting the scope of the invention.

### EXAMPLES

a) Increased Oil Production

The oil content of DDGS is sometimes higher than desired and methods of recovering more oil as a separate by-product for use in biodiesel production or other bio renewable products are sought. Much of the work in oil recovery from fermentation processes has focused on improving the extractability of the oil from the whole stillage. As mentioned in the specification, a better de-oiling leads to a better oil separation within the ethanol process. The corn oil production is a high valuable byproduct for the food and feed industry also for the Biodiesel production.

For that example the de oiling capability of the whole stillage during the centrifugation was tested and determined.

Four different setups were tested.

In the first fermentor (Fermentor#1) the fermentation cultivation was not treated with enzymes (0 g/t xylanase; 0 g/t pectinase), in the second, third and forth fermentor (Fermentor#2 to #4) the fermentation cultivation was treated with the enzyme composition comprising a xylanase and a pectinase from the beginning of the fermentation in different concentrations (from 25 g/t to 200 g/t xylanase and from 25 g/t to 200 g/t pectinase).

The trials were performed in 2 L fermentation scale. After the distillation the fermenter samples with the whole stillage were taken and the whole stillage was centrifuged to obtain thin stillage and the wet cake. Oil extraction was carried out by hexane and the mass of the extracted oil was related to the volume of thin stillage.

In one embodiment, the process of the production of ethanol from corn was performed as follows:

A) Process for producing fermentation products

a) Reducing the particle size of the starch-containing material by milling
corn (Company Pannonia, Hungary) was milled to <2 mm particle size (coffee mill, company Brunn)

b) Forming a slurry comprising the starch-containing material and water
1,5 kg milled corn was added to 4,96 L ml warm tap water (water hardness 3,57 mmol/L) at 35° C. to obtain a 25% solid solution with a final volume of 6 L in a Biostat C fermentor (company Sartorius) leading to a pH of about 5,6.

c) Liquefying of the starch-containing material
temperature was increased to 90° C.
1 ml α-amylase “α-amylase VF-Kartoffel” (Schliessmann, Nr. 5049) was diluted in 10 ml tap water and then the diluted amylase was added to the slurry
temperature was increased to 90° C.
the fermentor was incubated for 90 min at 90° C. and 450 rpm
the slurry was cooled to 30° C., pH was adjust to ˜4 with 30% H₂SO₄

d) Saccharifying of the liquefied material obtained
1,5 ml glucoamylase “Amylase GA 500” (Schliessmann, Nr. 5042) was diluted in 10 ml sterile tap water and then the diluted glucoamylase was added to the slurry, which is the saccharified liquefied material.

e) Fermentation
1,8 g (NH4)2 SO4 (i.e. 300 ppm ammonium sulphate) was added to the 6 L saccharified liquefied material.
the saccharified liquefied material containing 300 ppm ammonium sulphate in the Biostat C fermentor was stirred with 800 rpm for 5 min to distribute everything evenly.
the saccharified liquefied material containing 300 ppm ammonium sulphate (i.e. mash) was distributed in 1500 g single portions into four 2 L Biostat B fermentors (company Sartorius) containing a horseshoe mixer.
Enzyme stock preparation: 2,5 g of the pectinase (Pec3) with 90349 U/mL and 2,5 g of the xylanase (Xy116) with 10027 U/mL were added into a 50 mL graduated cylinder and filled to 50 mL with tap water.

The enzyme stock was transferred into a 50 mL tube and then stored at 4° C. until use within one hour.
The following volumes of the enzyme stock preparation were added to the 2 L Biostat B fermentor containing 1500 g of the saccharified liquefied material containing 300 ppm ammonium sulphate.

Fermentor #1: 0 mL of the enzyme stock preparation leading to 0 g/t of pectinase and 0 g/t of xylanase

Fermentor #2: 0,75 mL of the enzyme preparation leading to 25 g/t of pectinase and 25 g/t of xylanase

Fermentor #3: 2,25 mL of the enzyme preparation leading to 75 g/t of pectinase and 75 g/t of xylanase

Fermentor #4: 6,00 mL of the enzyme preparation leading to 200 g/t of pectinase and 200 g/t of xylanase
Yeast propagation: 300 ml autoclaved YNB (yeast nitrogen base) medium plus glucose with 10 g/L glucose medium resulting in pH 5,7 in a 1 L cultivation flasks, which had been inoculated with 2 ml yeast (Ethanol RED, company Fermentis) from a −80° C. cryo stock containing 20% glycerol, were incubated for 23 hours (30° C., 150 rpm) leading to the yeast culture.
Each of the four fermentors (Fermentor #1 to #4) was inoculated by 60 mL of the yeast culture.
Cultivations of the fermentors were carried out at 30° C. at 150 rpm, without pH control for 92,5 hours.
7-ml samples were taken twice the day by cut off pipette to monitor fermentation progress (ethanol concentration). The samples were transferred in 15 mL tubes and centrifuged at 4470 g for 10 minutes at 4° C. and stored until further analysis at −20° C.

f) Ending the fermentation process and distillation of ethanol
To end the fermentation process and to distillate ethanol the four fermentors were incubated at 80° C. for 20 min and stirred at 150 rpm.

g) Taking samples for analysis of oil release
After the 20 min at 80° C. and stirring at 150 rpm 3×40 to 45 ml samples were taken from each fermentor by a 50 mL pipette and filled into 50 mL tubes (samples of whole stillage).

B) Enzyme product activity determination:

DNSA solution: For the DNSA solution the following compounds were used:
5,00 g 3,5-Dinitrosalicylic acid (DNSA) was dissolved in 300 ml distilled H₂O.
add 50 ml NaOH/KOH-solution (4 M KOH+4 M NaOH) drop per drop
add 150 g K—Na-tartrate tetrahydrate
cool solution to room temperature
ad with distilled H₂O to 500 ml final volume
store in the darkness

a) Pectinase

Substrates: Polygalacturonic acid (Sigma 81325)

Substrates were dissolved in buffer to a concentration of 0,8% (w/v)

Buffer: 50 mM sodium acetate, pH 4,5

For the assay 96 well PCR microtiter plates (company Greiner) were used. The enzymes were diluted in buffer. 90 μl substrate and 10 μl enzyme solution were mixed. A blank was measured replacing enzyme solution with water. Incubation was carried out for 30 min at 37° C., followed by a 5 minute enzyme inactivation step at 99° C. and followed by cooling for 10 min at 4° C. In a second 96 well PCR microtiter plates (company Greiner) 50 μl of the incubated substrate-enzyme mix was incubated with 50 μl of the DNSA solution at 98° C. for 10 minutes and then cooled to 4° C. and incubated for 5 minutes at 4° C.

100 μl of the reaction was transferred into a well of 96 well transparent, flat bottom micro titer plate and the adsorption was measured at 540 nm by a micro titer plate reader (Tecan M1000).

b) Xylanase

Substrates: Xylan from birchwood (Sigma X0502)

Substrate was dissolved in buffer to a concentration of 1,5% (w/v)

Buffer: 100 mM sodium acetate, pH 5,0 containing 20 mM CaCl₂ and 0,4g/L Tween20

For the assay 96 well PCR microtiter plate (company Greiner) were used. The enzymes were diluted in buffer. 90 μl substrate and 10 μl enzyme solution were mixed. A blank was measured replacing enzyme solution with water. Incubation was carried out for 20 min at 40° C., followed by a 5 minute enzyme inactivation step at 99° C. and followed by cooling for 5 min at 4° C. 45,5 μL of the DNSA solution was added to the 96 well PCR microtiter plate by a multidrop (company Fisher-Scientific) and then the plate was incubated for 98° C. for 10 minutes and cooled to 4° C. and incubated for 5 minutes at 4° C.

100 μl of the reaction was transferred into well of a new 96 well transparent, flat bottom micro titer plate and then the adsorption was measured at 540 nm by a micro titer plate reader (Tecan M1000).

The activity is calculated as Units per μl or mg of enzyme product. 1 unit is defined as the amount of formed reducing ends in μmol per minute. The enzyme activities are shown in Table 1.

TABLE 1

| Type | Activity | Shortname |
|---|---|---|
| pectinase | 90349 U/mL | Pec3 |
| xylanase | 10027 U/mL | Xyl16 |

### Example 1

Analysis of De-Oiling

After the fermentation process and distillation of ethanol the following steps were made to analyze de-oiling:
1. About 40 ml of the samples of whole stillage were transferred into a 50 mL Falcon tube (FT1) and the mass was determined (mass FT1sample). The mass of the empty FT1 was previously determined (mass FT1empty). FT1 with the sample (FT1sample) was centrifuged at 3000 g for 30 minutes.
2. The supernatant was decanted into a new 50 mL Falcon tube (FT2) leading to the thin stillage and the mass was determined (mass FT2sample). The mass of FT2 was previously determined (mass FT2empty).
3. Falcon tubes FT1 and FT2 were stored at 4° C., until further analysis
4. Falcon tubes FT1 and FT2 were warmed to 20° C. in a water bath
5. To the sediment in FT1 5mL hexane was added, FT1 was washed with it and the liquid was poured into a round bottom flask (RBF1), which mass was previously determined (mass RBF1empty)
6. 10 mL of hexane was added to the supernatant in FT2 and shaken vigorously for two minutes.
7. FT2 was centrifuged at 3000 g for 10 minutes and the upper phase was added to RBF1
8. 2 mL of hexane was rinsed into FT2 and then added to RBF1
9. Steps 6 to 7 were repeated, but liquid was added into a new round bottom flask (RBF2) (mass RBF2empty).
10. Hexane in RBF1 and RBF2 was evaporated in a rotary evaporator at 250 mbar at 90° C.
11. RBF1 and RBF2 were dried at 90° C. for 12 hours.
12. RBF1 and RBF2 were cooled to room temperature and then the mass of RBF1 (mass RBF1oil) and RBF2 (mass RBF2oil) containing corn oil was determined

Calculation of extracted oil:

Mass of extracted oil [mg]=(mass RBF1oil−mass RBF1empty)+(mass RBF2oil−mass RBF2empty)

Calculation of sample mass:

Mass of thin stillage [mg]=(mass FT2sample−mass FT2empty)

Calculation of extracted oil per mass thin stillage

Extracted oil mass per mass of thin stillage [mg oil/mg thin stillage]=mass of extracted oil [mg]/mass of thin stillage [mg]

Calculation of extracted oil per volume thin stillage

Extracted oil mass per mass of thin stillage [mg oil/mg thin stillage] was calculated to extracted oil mass per mass of thin stillage [mg oil/kg thin stillage].

With the assumption that 1 kg thin stillage has 1 L volume:

Extracted oil mass per mass of thin stillage [mg oil/kg thin stillage] was converted to extracted oil per volume of thin stillage [mg oil/L thin stillage]

After the reaction of the enzyme composition comprising the main activities xylanase and pectinase with the concentrations 25 g/t, 75 g/t and 200 g/t of each compound followed by the centrifugation (conditions: 3000 rpm for 30min.) of the whole stillage an improved oil separation in the thin stillage was observed.

Table 2 is showing the extracted oil after centrifugation.

TABLE 2

| added | added | Extracted oil per | |
|---|---|---|---|
| xylanase | pectinase | volume of thin stillage | Related to control |
| [g/t] | [g/t] | [mg oil/L thin stillage] | [0 g/t = 100%] in [%] |
| 0 | 0 | 993 | 100 |
| 25 | 25 | 1116 | 112 |
| 75 | 75 | 1804 | 182 |
| 200 | 200 | 2272 | 228 |

The whole stillage, in which the inoculated mash was treated from the beginning of the fermentation with the enzyme composition comprising (Table 1) the main activities xylanase and pectinase activity, showed a higher extraction of oil per volume of thin stillage compared to the inoculated mash, which was not treated with enzymes.

1. A method of recovering oil, comprising (a) converting a starch-containing material into dextrins with an alpha-amylase; (b) saccharifying the dextrins using a carbohydrate source generating enzyme to form a sugar; (c) fermenting the sugar in a fermentation medium into a fermentation product using a fermenting organism, wherein the fermentation medium comprises a xylanase and a pectinase; (d) distilling the fermentation product to form a whole stillage; (e) separating the whole stillage into thin stillage and wet cake; and (f) recovering the oil from the thin stillage.

2. The method according to claim 1, wherein the fermentation product is selected from the group consisting of an acid, an alcohol, and hydrogen.

3. The method according to claim 2, wherein the alcohol is selected from the group consisting of ethanol, butanol, propanol, methanol, propanediol, and butanediol.

4. The method according to claim 2, wherein the acid is selected from the group consisting of lactic acid, propionic acid, acetic acid, succinic acid, malic acid, butyric acid, and formic acid.

5. The method of according to claim 1, wherein the starch containing material is obtained from cereals and/or tubers.

6. The method of according to claim 1, wherein the starch containing material is selected from the group groups consisting of corn, wheat, barley, rye, millet, sorghum, and milo.

7. The method of according to claim 1, wherein the microorganism is selected from the group consisting of a bacteria, a yeast, and a fungi.

8. A process for producing recoverable oil from fermentation processes, wherein the process sequentially comprises the following steps:
a) milling whole grain; b) liquefying the gelatinized milled whole grain, in the presence of an alpha-amylase; c) saccharifying the liquefied material in the presence of a glucoamylase; d) fermentation with a microorganism; e) distillation of fermented and saccharified material, thereby providing an ethanol fraction, wherein the liquefied mash is subjected to an effective amount of enzyme activity of a xylanase and a pectinase.

9. The process of claim 8, wherein the microorganism is a bacteria, yeast or fungi.
